(19) United States
(12) Patent Application Publication
(10) Pub. No.: US 2014/0171766 A1
(43) Pub. Date: Jun. 19, 2014

(54) OXIMETRY SENSOR ADJUNCT FOR ROUTINE DIAGNOSTIC SCREENING AND MONITORING

(71) Applicant: SPOT CHECK MEDICAL LTD. CO., A NEW MEXICO LLC, (US)

(72) Inventor: Craig A. Ferris, Fallbrook, CA (US)

(73) Assignee: SPOT CHECK MEDICAL LTD. CO., A NEW MEXICO LLC, Fallbrook, CA (US)

(21) Appl. No.: 13/713,314

(22) Filed: Dec. 13, 2012

(51) Int. Cl. A61B 5/1455; A61B 5/00

(52) U.S. Cl. CPC A61B 5/14552; A61B 5/6833
USPC 600/323

(57) ABSTRACT

Provided herein is an invention of a disposable adjunct developed to address limitations associated with oximetry sensor use: patient skin irritation and breakdown; compromised or interrupted display of data resulting from ambient light interference; costs incurred from replacing sensors with failed adhesion; and use of extra sensors for acute monitoring of multiple patient sites. This adjunct may be manufactured at a fraction of the cost of traditional oximetry sensors and can be retrofit to any existing sensor. Data aberrations caused by ambient light interference are reduced, utilizing a photo-opaque layer to prevent interference from above and restoring adhesion with a hydrocolloid adhesive layer to prevent interference from below. The hydrocolloid adhesive layer provides an alternative to relatively aggressive adhesives found on the majority of oximetry sensors, thereby improving patient tolerance through reduced skin irritation. Additional intermediate layers may also be provided.

### FIELD OF THE INVENTION

The present invention is generally directed to devices and methods for determining blood oxygen saturation in various tissues and organs of living creatures, especially humans. One application of the invention is particularly directed to improving the longevity, adhesion integrity, and patient tolerance of existing sensors used for oximetry purposes.

### BACKGROUND

Oximetry refers to the measuring of oxygen saturation of the blood by means of an oximeter. Traditional oximeters may be designed for cerebral or arterial oximetry and include an energy-emitting component and an energy-detecting sensor. Typically the energy-emitting component is a light emitting diode (LED) or light emitting optode and the energy-detecting sensor is a photodiode or photo-optode. Spectrophotometric oximeters that rely on the 700 to 1300 nm wavelength range of near infrared spectrum (i.e. NIRS oximeters) and use NIRS sensors have been particularly useful, in part due to the divergent absorption spectra illustrated by oxygenated and deoxygenated hemoglobin and by oxidized and reduced cytochrome. Absorption of near infrared energy is caused by hemoglobin, an oxygen-carrying medium in blood, and cytochrome which performs oxidation-reduction reactions in cells.

Cerebral oximetry sensors tend to differ from arterial oximetry sensors due to the different conditions encountered in each sensing location. Arterial pulse oximeters monitor primarily highly oxygenated arterial blood and require a pulse while cerebral oximeters monitor mixed venous arterial blood with no need for pulsatile flow through the vessels. Cerebral oximetry requires deeper signal penetration to access brain tissue; amplified sufficiently to first traverse layers of skin and bone. Such is also achieved by widening separation of the light sources and detectors in accordance with the Beer-Lambert Law. Accordingly, due to the greater attenuation of light from greater separation distances in cerebral oximetry the detected light intensities are very small, on the order of picoamps. As cerebral oximetry measures oxygen saturation in the venous range, a steeper area of the oxygen dissociation curve, signal aberrations are magnified in the form of greater data deviations. Cerebral oximetry sensors must therefore be more sensitive than arterial or pulse oximetry sensors and can be more vulnerable to signal noise and interference.

Traditional oximetry sensors are underutilized and the market is underdeveloped due to the high costs of the sensors and lack of reimbursement from most health insurance plans, at least for prophylactic purposes. For example, the average NIRS sensor price is between $80 and $200 per sensor. Sensing at multiple sites on a single patient is often desirable but at these prices could cost over $800. NIRS spectrophotometry has numerous applications as it can detect crisis states early to prevent permanent damage, alerting medical personnel to serious dangers before it is too late. Such applications include monitoring specific organs or tissues during surgery, ambulatory screening, emergency room screening, critical care monitoring, and neo-natal and pediatric screening and monitoring, among others. If the cost of NIRS sensors relative to their lifespan was more affordable they could be utilized more widely to the benefit of a vast underserved population.

The cost of traditional sensors is problematic especially as it relates to sensor lifespan. In addition to legal limitations on the number of uses in the form of manufacturer indications, warranties, and user agreements, clinicians will find the sensors themselves are functionally limited as they do not hold up well over time. Problems include sensor adhesive breakdown, skin irritation from the adhesive, and increasing inaccuracies due to ambient light entering from above and underneath in regions where the adhesive has broken down. It would be desirable to provide a device that addresses all of these issues to improve the NIRS sensor life to cost ratio enabling more patients to benefit from the insight provided by NIRS sensors to prevent the irreversible results of oxygen deprivation. The present invention meets these and other needs.

### SUMMARY OF THE INVENTION

One of several objectives of the present invention is to provide a device for use with any of the existing NIRS sensors that prolongs sensor life, restores adhesion integrity, increases patient tolerance by reducing skin irritation, and makes NIRS sensor use more affordable.

To accomplish this objective, the device includes an adjunct that easily attaches to existing NIRS sensors and can be specially adapted or modified to fit a particular sensor. The adjunct relies upon existing sensors and monitors and does not require electronics of its own. The adjunct may be designed to fit any variety of existing sensors for any part of the body, including cerebral oximetry sensors (e.g. on the head) and possibly pulse oximetry sensors (e.g. on a finger, ear lobe, etc.)

In another variation the device may be created with its own electronics so that it stands alone with advantages being realized from the photo-opaque cover and improved hydrocolloid adhesive patient contact layer.

In addition to serving the needs of the surgical patient with its enhanced benefit to cost ratio the present invention may also find utility for the average patient for routine screening purposes.

According to a first aspect of the present invention an adjunct for retrofitting to an oximetry sensor is provided to improve at least one of the useful life, adhesion integrity, and patient tolerance thereof. The adjunct includes an adhesive patient contact layer in which a first side of the adhesive patient contact layer is configured to attach to a patient's skin and a second side of the adhesive patient contact layer is configured to attach to an oximetry sensor, directly or through an intermediate layer; and a photo-opaque layer for application over the oximetry sensor. The adhesive patient contact layer is configured to transmit energy from an energy-emitting component and to an energy-detecting component of the oximetry sensor. The adhesive patient contact layer may have at least one aperture therein, such that when retrofitted to the oximetry sensor the aperture is aligned with at least one of an energy-emitting component and an energy-detecting component of the oximetry sensor to facilitate energy from the energy-emitting component or energy to the energy-detecting component passing through the aperture. The adhesive patient contact layer may include a hydrocolloid. The hydrocolloid adhesive patient contact layer may have a thickness of 10 to 30 mil (1 mil=1/1000 of an inch). The photo-opaque layer may include polyethylene terephthalate (PET).

The adjunct may include at least one intermediate layer between the adhesive patient contact layer and the oximetry sensor. The adjunct may include two intermediate layers between the adhesive patient contact layer and the oximetry sensor, a barrier layer in direct contact with the adhesive patient contact layer, and a double-sided adhesive layer in direct contact with the oximetry sensor. The intermediate barrier layer may include polypropylene. The intermediate barrier layer may have a thickness of 1 to 2 mil.

The adjunct may further include a label liner layer above the intermediate layer and a liner layer below the adhesive patient contact layer, in which the liner layer is removed in order to attach the adhesive patient contact layer to a patient and the label liner layer is removed in order to attach the intermediate layer to the oximetry sensor.

The energy-emitting component of the oximetry sensor may be a near infrared spectrum light emitting diode or a near infrared spectrum light emitting optode. The energy-detecting component of the oximetry sensor may be a near infrared spectrum photodiode or a near infrared spectrum photo-optode.

According to a second aspect of the present invention a dispensing roll is provided having a plurality of sheets along the roll, the plurality of sheets being separated from each other by perforations that permit one sheet to be removed from adjacent sheets, each sheet of said plurality of sheets having the adjunct for retrofitting to an oximetry sensor, as described in any of the preceding paragraphs, disposed thereon. For the dispensing roll the adjunct may be disposed on each sheet in two sections, a first section including the adhesive patient contact layer and a second section including the photo-opaque layer. Alternatively, the adjunct may be disposed on each sheet as one section including the adhesive patient contact layer or the photo-opaque layer.

According to a third aspect of the present invention a method is provided for improving at least one of useful life, accuracy, and patient tolerance of an oximetry sensor and reducing replacement cost thereof. The method includes applying an adhesive patient contact layer to a skin surface of a patient, the adhesive patient contact layer being separate from an oximetry sensor; applying the oximetry sensor above the adhesive patient contact layer, directly or indirectly through intermediate layers; and/or applying a photo-opaque layer over the oximetry sensor. The method may also include sensing oxygen saturation with the oximetry sensor through the adhesive patient contact layer.

According to a fourth aspect of the present invention an improved oximetry sensor is provided. The improved oximetry sensor includes an adhesive patient contact layer of a hydrocolloid, a photo-opaque layer, and an energy-emitting component and an energy-detecting component, the energy-emitting component and the energy-detecting component both positioned between the hydrocolloid adhesive patient contact layer and the photo-opaque layer.

Other features and advantages of the present invention will become apparent from the following detailed description, taken in conjunction with the accompanying drawings, which illustrate, by way of example, the principles of the invention.

### BRIEF DESCRIPTION OF THE DRAWINGS

FIG. 1 is a top view of an adjunct according to an embodiment of the present invention disposed on sheets of a dispensing roll, including an expanded view of the individual layers in the lower multilayer piece.

FIG. 2 is a side view of a patient's skin with an adjunct and oximetry sensor thereon, as used according to an embodiment of the present invention.

### DETAILED DESCRIPTION OF THE PREFERRED EMBODIMENTS

Briefly, and in general terms, provided herein is a medical device in the form of an adjunct that can be retrofitted to existing sensors for measuring oxygen saturation and optionally, other physiologic data in various parts and tissues of the body. The adjunct will find usefulness in the emergency room, surgery room, and for critical care, neonatal, and pediatric screening, among countless other applications. By extending the performance life of existing sensors, restoring adhesion integrity, and improving patient tolerance through superior adhesives the adjunct described herein makes it affordable to measure oxygen saturation at a greater number of sites in a greater number of patients.

According to one embodiment, the medical device takes the form of an adjunct and requires existing sensors (e.g. NIRS sensors) to function. The adjunct itself does not have any electronic components, energy-emitters, or energy-detectors. Thus, all of the electronics required by monitors (e.g. NIRS monitors) are found on the conventional sensor used with the adjunct. In effect, the adjunct forms a sandwich around the sensor, improving contact and sealing against patient skin underneath and blocking out ambient energy noise from above. By eliminating electronic components from the adjunct it can be manufactured for substantially less than the cost of a traditional sensor.

Over time the functionality of conventional sensors is impaired as they suffer from adhesive breakdown, ambient light interference from above and below, and they cause skin irritation. Makeshift remedies to improve adhesion are imperfect, messy, tedious, and time-consuming. After several uses the electronics (e.g. light emitting/detecting diodes/optodes) of existing sensors are typically intact and not responsible for the impaired functionality. Perfectly good sensors are discarded simply because they are not performing correctly, due to the three reasons listed above that have nothing to do with electronics. With the adjunct described herein (and marketed under the trade name SPOT CHECK™) existing sensors may be resurfaced for continued use at a fraction of the cost of a new sensor. The ability to provide affordable, routine diagnostic screening or “spot checking” for newly admitted patients is made possible because of the reduced price of the adjunct compared to a brand new traditional sensor.

Existing sensors and monitors with which the adjunct of the present invention will work include but are not limited to those provided by widely recognized companies in the field of NIRS sensing and monitoring, including Covidien PLc, CAS Medical and Nonin Corp. The adjunct shown and described herein would have different versions to fit the different manufacturer's sensors.

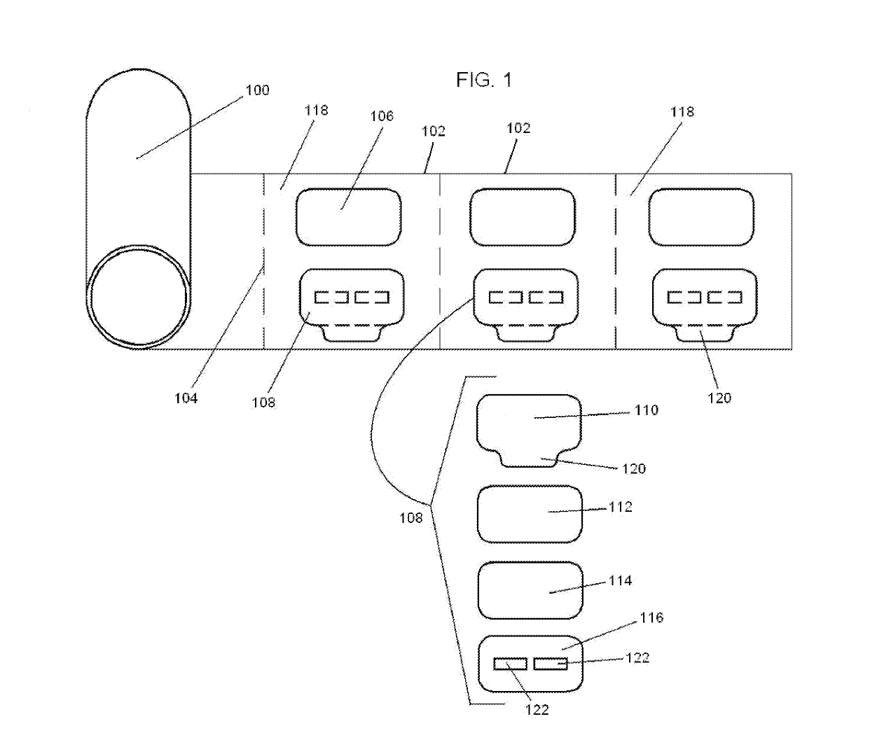

As shown in FIG. 1, the adjunct layers may be dispensed on a roll 100 including individual sheets 102 that can be torn off by way of perforations 104 in between adjacent sheets. Each sheet contains all layers necessary for a complete adjunct. The layers may be in two separate units, a first unit including only the photo-opaque layer 106 and a second unit 108 including all the other layers. That is, the second unit includes a sandwich of a top label liner layer 110, one or more intermediate layers (e.g. double-sided adhesive layer 112 and barrier layer 114), and the adhesive patient contact layer 116 already adhered to each other.

The base 118 of the sheet 102 facing the bottom of the two units 106, 108 can be made of any material that provides good release characteristics, including for example a siliconized paper or some similar polymer material.

The photo-opaque layer 106 may be made of any substance that will block out a sufficient amount of ambient light energy from above the oximetry sensor thus reducing interference. For example but not by way of limitation the photo-opaque layer may be polyethylene terephalate (PET) tape or a flexible polyester film, such as that available under the tradename MYLAR®. The photo-opaque layer 106 may include a logo, directions, or manufacturing information (e.g. date, serial no., etc.).

In the lower second unit 108 of the sheet, composed of a plurality of layers, at the top the label liner layer 110 may include a logo, directions, manufacturing info. (date, serial no., etc.). This top label liner layer 110 can be peeled off through use of the tab 120 without contaminating the upper adhesive surface of the double-sided adhesive layer 112 below.

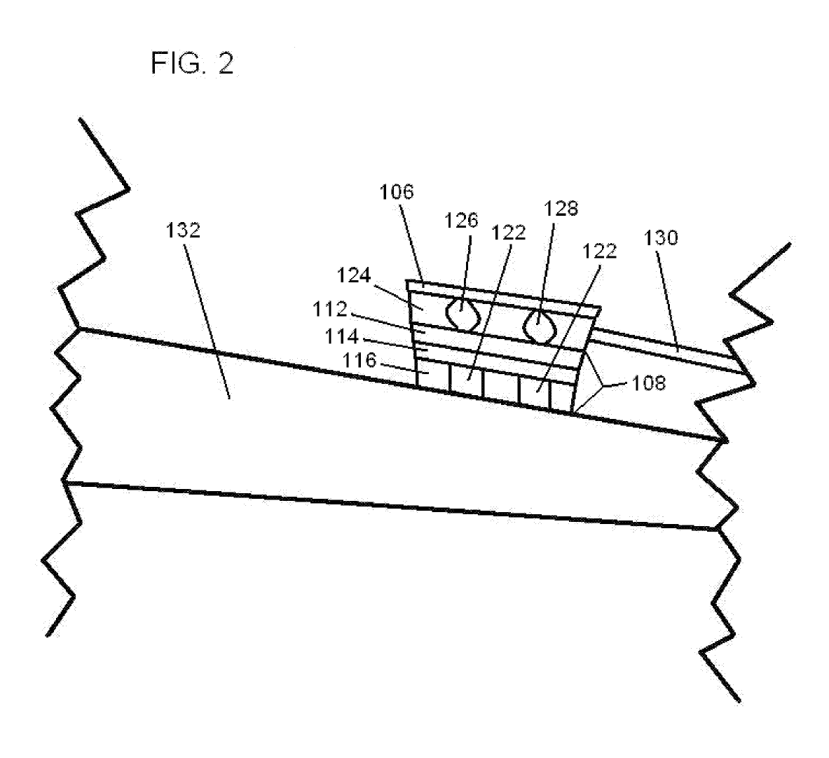

On the sheet 102, the thin, double-sided adhesive layer 112 adheres to both the label liner layer 110 above and the barrier layer 114 below. In practice the conventional oximetry sensor 124 is attached to this upper adhesive surface of the double-sided adhesive layer as shown in FIG. 2. Once the protective label liner layer 110 is removed, the double-sided adhesive layer 112 may connect to the sensor. Another purpose of the double-sided adhesive layer is to permit detachment of the sensor in a manner such that the sensor may be detached and reattached to this or another spot check unit.

The barrier layer 114, below the double-sided adhesive layer 112, physically isolates the sensor 124 from the patient 132, in the event there are concerns about cross-contamination while using the sensor on multiple sites. The barrier layer may be clear polypropylene of 1 to 2 mil in thickness.

All layers may be adapted and modified as desired for specific applications. However, it is contemplated that materials currently approved for and utilized in the manufacture of medical disposables and determined by the OEM to provide the appropriate adhesion and release characteristics will be suitable.

FIG. 2 shows a side view of the adjunct on top of the skin 132 of a patient with the sensor 124 between the two units 106, 108 of the adjunct, the lower set of layers below the sensor including the adhesive patient contact layer 116, the barrier layer 114, and the double-sided adhesive layer 112, and the upper layer above the sensor including the photo-opaque layer 106. The top label liner layer has been removed in order to secure the sensor to the double-sided adhesive layer. In practice, generally the second unit 108 should be removed from the sheet first and the adhesive patient contact layer secured to the skin surface of a patient before peeling off the top label liner layer to attach the sensor.

As the sensor 124 is attached above the double-sided adhesive layer 112, the electronics of the sensor must be capable of functioning through the double-sided adhesive layer 112, the barrier layer 114 beneath, and the adhesive patient contact layer 116 further beneath. More specifically, energy from the energy-emitting component of the sensor (e.g. energy-emitting optode) must be able to transmit through each of these layers to reach the patient's body. Reflected energy must be able to travel back through each of these layers to reach the energy-detecting component of the sensor (e.g. photo-optode) without being overly attenuated such that is difficult to detect and measure with accuracy.

Typically the adhesive patient contact layer 116 is thicker than each of the barrier layer 114 and double-sided adhesive layer 112 above it. As shown in FIGS. 1 and 2, according to one embodiment, cut-out apertures 122 in the adhesive patient contact layer 116 aligned with the energy pathways for the energy-emitting component 126 and energy-detecting component 128 of the oximetry sensor 124 allow energy to travel more easily without being diminished or attenuated to the extent that would occur if the energy had to travel through the entire solid adhesive patient contact layer. The oximetry sensor 124 attaches to a monitor (not shown) through a cable 130 or wirelessly.

According to one embodiment of the present invention, the adjunct is used with a sensor device that senses oxygen saturation in tissues using near infrared spectrometry. However, the invention is not so limited and it is contemplated that the adjunct shown and described herein may also be used with other medical devices that measure other types of physiologic data in a similar manner within the spirit and scope of the present invention. For example, by improving adhesion, patient tolerance, and substantially reducing ambient noise the adjunct may be used to resurface and extend the useful life of other types of electronic medical devices while eliminating cross-contamination concerns.

According to another embodiment of the present invention an improved self-sufficient sensor may be provided with electronics by incorporating the photo-opaque shield layer and hydrocolloid patient adhesive layer with its superior biocompatibility, adhesion, and release characteristics.

The present invention is not limited to the embodiments described above. Various changes and modifications can, of course, be made, without departing from the scope and spirit of the present invention. Additional advantages and modifications will readily occur to those skilled in the art. Accordingly, various modifications may be made without departing from the spirit or scope of the general inventive concept as defined by the appended claims and their equivalents.

What is claimed are:

1. An adjunct for retrofitting to an oximetry sensor to improve at least one of useful life, restored adhesion integrity, and patient tolerance thereof, comprising:
an adhesive patient contact layer, wherein a first side of the adhesive patient contact layer is configured to attach to a patient's skin and a second side of the adhesive patient contact layer is configured to attach to an oximetry sensor, directly or through an intermediate layer; and
a photo-opaque layer for application over the oximetry sensor.

2. The adjunct of claim 1, wherein the adhesive patient contact layer is configured to transmit energy from an energy-emitting component and to an energy-detecting component of the oximetry sensor.

3. The adjunct of claim 1, wherein the adhesive patient contact layer has at least one aperture therein, such that when retrofitted to the oximetry sensor the aperture is aligned with at least one of an energy-emitting component and an energy-detecting component of the oximetry sensor to facilitate energy from the energy-emitting component or energy to the energy-detecting component passing through the aperture.

4. The adjunct of claim 1, wherein the adhesive patient contact layer comprises a hydrocolloid.

5. The adjunct of claim 4, wherein the hydrocolloid adhesive patient contact layer has a thickness of 10 to 30 mil.

6. The adjunct of claim 3, wherein the adhesive patient contact layer comprises a hydrocolloid.

7. The adjunct of claim 6, wherein the hydrocolloid adhesive patient contact layer has a thickness of 10 to 30 mil.

8. The adjunct of claim 1, wherein the photo-opaque layer comprises polyethylene terephthalate (PET).

9. The adjunct of claim 1, further comprising at least one intermediate layer between the adhesive patient contact layer and the oximetry sensor.

10. The adjunct of claim 9, further comprising two intermediate layers between the adhesive patient contact layer and the oximetry sensor, a barrier layer in direct contact with the adhesive patient contact layer, and a double-sided adhesive layer in direct contact with the oximetry sensor.

11. The adjunct of claim 10, wherein the intermediate barrier layer comprises polypropylene.

12. The adjunct of claim 11, wherein the intermediate barrier layer has a thickness of 1 to 2 mil.

13. The adjunct of claim 9, further comprising a label liner layer above the intermediate layer and a liner layer below the adhesive patient contact layer, wherein the liner layer is removed in order to attach the adhesive patient contact layer to a patient's skin and the label liner layer is removed in order to attach the intermediate layer to the oximetry sensor.

14. The adjunct of claim 2, wherein the energy-emitting component of the oximetry sensor is selected from the group consisting of: a near infrared spectrum light emitting diode and a near infrared spectrum light emitting optode.

15. The adjunct of claim 2, wherein the energy-detecting component of the oximetry sensor is selected from the group consisting of: a near infrared spectrum photodiode and a near infrared spectrum photo-optode.

16. A dispensing roll comprising a plurality of sheets along the roll, the plurality of sheets being separated from each other by perforations that permit one sheet to be removed from adjacent sheets, each sheet of said plurality of sheets having the adjunct of claim 1 disposed thereon.

17. The dispensing roll of claim 16, wherein the adjunct is disposed on each sheet in two sections, a first section comprising the adhesive patient contact layer and a second section comprising the photo-opaque layer, or in one section comprising the adhesive patient contact layer or the photo-opaque layer.

18. A method for improving at least one of useful life, restored adhesion integrity, and patient tolerance of an oximetry sensor and reducing replacement cost thereof, comprising:
applying an adhesive patient contact layer to a skin surface of a patient, the adhesive patient contact layer being separate from an oximetry sensor;
applying the oximetry sensor above the adhesive patient contact layer, directly or indirectly through intermediate layers; and
applying a photo-opaque layer over the oximetry sensor.

19. The method of claim 18, further comprising sensing oxygen saturation with the oximetry sensor through the adhesive patient contact layer.

20. An oximetry sensor comprising:
an adhesive patient contact layer comprising a hydrocolloid;
a photo-opaque layer; and
an energy-emitting component and an energy-detecting component, the energy-emitting component and the energy-detecting component both positioned between the hydrocolloid adhesive patient contact layer and the photo-opaque layer.
